(19) United States
(12) Patent Application Publication
(10) Pub. No.: US 2013/0281884 A1
(43) Pub. Date: Oct. 24, 2013

(54) MEASUREMENT AND RESULTING COMPENSATION OF INTRAMEDULLARY NAIL DEFORMATION

(71) Applicant: Vector Sight Inc., Germantown, TN (US)

(72) Inventors: Michael W. Mullaney, Kinnelon, NJ (US); Stephen T. Miller, Scotts Valley, CA (US); William Brian Austin, Germantown, TN (US); Todd A. Martens, Denver, CO (US)

(73) Assignee: Vector Sight Inc., Germantown, TN (US)

(21) Appl. No.: 13/868,759

(22) Filed: Apr. 23, 2013

Related U.S. Application Data
(60) Provisional application No. 61/637,405, filed on Apr. 24, 2012.
(60) Provisional application No. 61/783,745, filed on Mar. 14, 2013.

(51) Int. Cl. A61B 5/107

(52) U.S. Cl. CPC A61B 5/107
USPC 600/587

(57) ABSTRACT

The present disclosure relates to systems and methods for measuring deformation of an orthopedic implant in a patient to identify a location of a feature of the orthopedic implant in the patient. Mechanical locking features may be used to align a drilling guide with the feature of the orthopedic implant.

### PRIORITY

This application claims the benefit of the filing date of U.S. Provisional Application No. 61/637,405, filed Apr. 24, 2012, titled “Measurement and Resulting Compensation of Intramedullary Nail Deformation,” and U.S. Provisional Application No. 61/783,745, filed Mar. 14, 2013, also titled “Measurement and Resulting Compensation of Intramedullary Nail Deformation,” both of which are incorporated herein by reference in their entirety.

### FIELD OF THE INVENTION

This application relates to systems and methods to aid in the location and the insertion of distal interlocking screws in intramedullary nailing procedures.

### BACKGROUND

The use of intramedullary nails to treat fractures of long bones has become very common. To aid in healing, an intramedullary nail is inserted across a fracture site to hold both ends of the fractured bone in relative proximity. It is then locked with interlocking screws to prevent rotational and axial motion at the fracture site. For reference purposes, the proximal end of the nail is the end in close proximity to the nail's entry into the medullary canal, and the distal end is the opposite end.

Mechanical fixtures help surgeons achieve proper alignment of the interlocking screws at the proximal end of the intramedullary nail. Surgeons utilize a combination of mechanical fixtures, x-ray guidance, and/or electromagnetic targeting devices in attempt to obtain proper alignment of the distal interlocking screws. However, use of mechanical fixtures for the distal interlocking portion of the procedure is challenging because significant deformation of the nail can occur upon implantation (Krettek, 1996). In general the intramedullary nail will deform as it travels down the patient's medullary canal to match the general shape of the bone it is being positioned in.

A typical approach to obtain proper orientation of the distal interlock screws is to use a free hand technique termed “perfect circles”. This approach utilizes images from a mobile fluoroscopy machine (c-arm) to orient the distal interlock holes such that the near and far end of the hole appear as a circle on the fluro image. If there is misalignment of the c-arm relative to the through-hole the 2D fluro image of the hole will appear oblong. The surgeon will use this trial and error technique to refine the position of the c-arm until the through-hole image appears as a perfect circle. The surgeon then aligns the drill to the orientation of the axis of the c-arm beam of x-rays and then drills through the bone/nail construct. This approach can be very time consuming as its dependent on both the surgeon's technique and the x-ray technician's ability. Additionally, this approach increases the x-ray exposure to the surgeon, operating room staff, and patient.

More recently, electromagnetic (EM) tracking devices have been utilized to assist the surgeon with this distal locking challenge. These devices require an EM receiver element positioned in the center of the intramedullary nail in close proximity to the distal holes. An EM field emitter is connected to a drill guide, and a computer program provides feedback to the surgeon regarding where the drill guide is in relation to the element, and thus the distal holes. One known downside to this approach is that the surgeon is forced to cross lock the nail distally first (because the EM receiver probe in the nail blocks the path of any proximally cross locking screws). Additionally, EM field distortions can induce inaccuracies into the system.

The present invention relates to a novel method of measuring the deformation of intramedullary nail and then compensating for the deformations to provide a safe and reliable method to distally lock an intramedullary nail.

### SUMMARY

In one exemplary aspect, the present disclosure is directed a probe for measuring deformation of an orthopedic implant implanted in a patient. The probe includes a body portion configured to be implanted into the orthopedic implant, the body portion being arranged to conform with deformations in the orthopedic implant, and the probe includes a deformation measuring element associated with the body portion in manner to measure deformation of the probe when inserted into an orthopedic implant.

In an aspect, deformation of the probe mimics deformation of the orthopedic implant. In an aspect, the deformation measuring element comprises one or more strain gages disposed in one or more locations on the probe. In an aspect, the probe is divided into a plurality of segments and the deformation measuring element is disposed to measure deformation of one of the plurality of segments. In an aspect, the deformation measurement element comprises one or more deformation measurement elements associated with each of the plurality of segments, and wherein the measured deformation is integrateable to determine a composite deformation of the orthopedic implant. In an aspect, each of the plurality of segments is demarcated with the demarcation elements configured to guide a portion of the probe so that the probe follows the same trajectory as the implant. In an aspect, the probe is sized and shaped to displace relative to the orthopedic implant such that a single deformation measuring element yields multiple data points. In an aspect, the probe is configured to measure deformation enabling various planes of deformation to be determined. In an aspect, the probe is configured to measure deformation enabling radial deflection to be determined in order that curvature and/or trajectories can be calculated. In an aspect, the body portion comprises a square cross-section. In an aspect, the deformation measuring element comprises strain gauges disposed on opposing sides of the square body portion.

In an exemplary aspect, the present disclosure is directed to a method of measuring deformation to align an instrument with an orthopedic implant implanted in a patient. The method includes providing a body portion of a probe in an orthopedic implant, the body portion being arranged to conform with deformations in the orthopedic implant, and the method includes measuring deformation of the probe with a deformation measuring element.

In an aspect, the method includes making adjustments to a targeting jig such that the orthopedic implant remains in alignment with the targeting jig when deflection is present in the orthopedic implant, the targeting jig, or both. In an aspect, the method includes dividing the body portion of the probe into segments; and wherein measuring deformation of the probe comprises measuring deformation of the segments in a piecewise manner. In an aspect, the method includes integrating the measured segments together to determine a composite deformation of the body portion of the probe. In an aspect, the method includes changing the position of the body portion of the probe relative to the orthopedic implant while measuring to yield multiple data points from a given deformation measuring element. In an aspect, changing the position of the body portion comprise rotating the body portion about an axis of the probe to obtain deformation information. In an aspect, the method includes using the obtain deformation information to determine various planes of deformation of the orthopedic implant. In an aspect, changing the position of the body portion comprises axially translating the body portion along an axis of the probe to obtain a collection of points representing radial deflection. In an aspect, the method includes using the collection of points to determine curvature or trajectory of the orthopedic implant. In an aspect, the method includes compensating for measured deflection of the orthopedic implant by adjusting a targeting jig to accurately target a certain feature of the orthopedic implant. In an aspect, adjusting a targeting jig to accurately target a certain feature comprises adjusting the jig in more than one plane that intersects the orthopedic implant.

In an exemplary aspect, the present disclosure is directed to a method of aligning a jig with a feature of an implant implanted in a patient. The method includes detecting deformation with a strain gage on an orthopedic implant, and the method includes based on the detected deformation, calculating the actual deformation to accurately predict a location of a feature on the orthopedic implant while the implant is in a deformed state.

In an aspect, the method includes aligning a jig with the feature on the orthopedic implant while the implant is in the deformed state taking into account the detected deformation. In an aspect, calculating the actual deformation comprises comparing detected deformation from a first probe associated with or incorporated into the implant and detected deformation from a second probe associated with or incorporated into the jig. In an aspect, the strain gage is disposed on a probe inserted into the orthopedic implant.

In an exemplary aspect, the present disclosure is directed to a system for determining the location of a feature of an implant and aligning a surgical instrument with the feature. The system includes a measuring element configured to measure deformation of an orthopedic implant in a patient to identify a feature of the orthopedic implant in the patient. The system also includes a jig dimensionally adjustable to match the measured deformation of the orthopedic implant to align with the feature of the orthopedic implant.

In an aspect, the jig has one or more degrees of freedom such that the jig can be manipulated into a desired position and, wherein the jig is configured to be locked into the desired position. In an aspect, the one or more degrees of freedom are achieved using a series of sliding elements. In an aspect, the series of sliding elements comprise rectangular blocks in rectangular recesses. In an aspect, the rectangular blocks and rectangular recesses comprise a first rectangular block and rectangular recess and a second rectangular block and rectangular recess, with the first rectangular block and rectangular recess arranged orthogonal to the second rectangular block and rectangular recess. In an aspect, the sliding elements comprise a rectangular tongue in a first rectangular slot. In an aspect, the sliding elements comprise the rectangular tongue in a second rectangular slot. In an aspect, the jig comprises three adjustable struts operable to align a surgical instrument in three degrees of freedom. In an aspect, the system includes a processing system configured to output adjustment settings for the three adjustable struts to align a portion of the jig with the feature of the orthopedic implant.

The present disclosure is directed to systems and methods that determine intramedullary nail deformation after the nail has been inserted into the medullary canal. Knowledge of the deformations in multiple planes allows calculations of new “distorted” positions of the distal holes. This information can be used with a jig or mechanical locking fixtures to accurately target distal locking holes of the intramedullary nail.

These systems use an instrumented probe that detects the deformation of the intramedullary nail. Information from this instrumented probe can provide the user information on how much to adjust the mechanical distal locking fixtures to accurately drill through the distal nail holes.

In some scenarios, once the deformations of the intramedullary nail are measured, the instrumented probe is removed from the intramedullary nail and docked in a flexible mechanical fixture/drill guide. With computer display providing instantaneous feedback, the user matches the deformations of the mechanical fixture/drill guide to that of the intramedullary nail to then accurately target the distal locking holes.

In yet other scenarios, the intramedullary nail deformation information works in conjunction with current navigation systems, either optical or EM, to provide quick and repeatable distal targeting guides.

Regardless of the approach, the systems and methods may reduce undesirable x-ray exposure to the patient, the surgeon, and the operating room staff all while providing a solution that does not alter the required surgical steps to locking an intramedullary nail (i.e. can lock either proximally or distally first depending on the surgeons desired approach).

It is to be understood that both the foregoing general description and the following detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTION OF THE DRAWINGS

The accompanying drawings illustrate embodiments of the devices and methods disclosed herein and together with the description, serve to explain the principles of the present disclosure.

FIG. 1 is an illustration of an exemplary intramedullary nail disposed within a femur of patient in accordance with one aspect of the present disclosure.

FIG. 2 is an illustration of the exemplary intramedullary nail of FIG. 1.

FIG. 3 is an isometric view of a probe assembly in accordance with one aspect of the present disclosure.

FIG. 4 is an illustration of a cross-sectional view of the exemplary intramedullary nail of FIG. 2 with a nail probe disposed in a central cavity therein in accordance with one aspect of the present disclosure.

FIG. 5 is an illustration of a cross-sectional view of an exemplary probe taken along lines 5-5 in FIG. 3.

FIG. 6 is an illustration of a cross-sectional view of another exemplary probe in accordance with one aspect of the present disclosure.

FIG. 7A is an illustration of a cross-sectional view of an exemplary probe taken along lines 7A-7A in FIG. 6.

FIG. 7B is a more detailed illustration of the probe of FIG. 6, showing the detail of FIG. 7B identified in FIG. 6.

FIG. 8 is an illustration of a top view of a jig forming a portion of an intramedullary nail implantation system in accordance with one aspect of the present disclosure.

FIG. 9 is an illustration of an elevation view of the jig of FIG. 8.

FIG. 10 is an illustration of a side view of the jig of FIG. 8.

FIG. 11 is an illustration of a side view of a jig forming a portion of another intramedullary nail implantation system in accordance with one aspect of the present disclosure.

FIG. 12 is an illustration of the jig of FIG. 11.

FIG. 13 is an illustration of a side view of a drill guide forming a portion of the intramedullary nail implantation system of FIG. 11 in accordance with one aspect of the present disclosure.

FIG. 14 is an illustration of the drill guide of FIG. 13.

FIG. 15 is an illustration of a perspective view of a jig forming a portion of another intramedullary nail implantation system in accordance with one aspect of the present disclosure.

FIG. 16 is an illustration of a side view of the jig of FIG. 15.

FIG. 17 is an illustration of an end view of the jig of FIG. 15.

FIG. 18 is an illustration of a partial cross-sectional view of the jig of FIG. 15.

FIG. 19 is a more detailed illustration of the jig of FIG. 18, taken along the callout FIG. 19 in FIG. 18.

FIG. 20 is a more detailed illustration of the jig of FIG. 18, taken along the callout FIG. 20 in FIG. 18.

FIG. 21 is an illustration of a cross-sectional view taken along the lines 21-21 in FIG. 20.

FIG. 22 is an illustration of a cross-sectional view taken along the lines 22-22 in FIG. 20.

FIG. 23 is an illustration of a cross-sectional view taken along the lines 23-23 in FIG. 20.

FIG. 24 is an illustration of a perspective view of a jig forming a portion of another intramedullary nail implantation system in accordance with one aspect of the present disclosure.

FIG. 25 is an illustration of a side view of the jig of FIG. 24.

FIG. 26 is an illustration of an end view of the jig of FIG. 24.

FIG. 27 is an illustration of an end view of the jig of FIG. 24.

FIG. 28 is an illustration of a partial cross-sectional view of the jig of FIG. 24 taken along the lines 28-28 in FIG. 27.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For simplicity, in some instances the same reference numbers are used throughout the drawings to refer to the same or like parts.

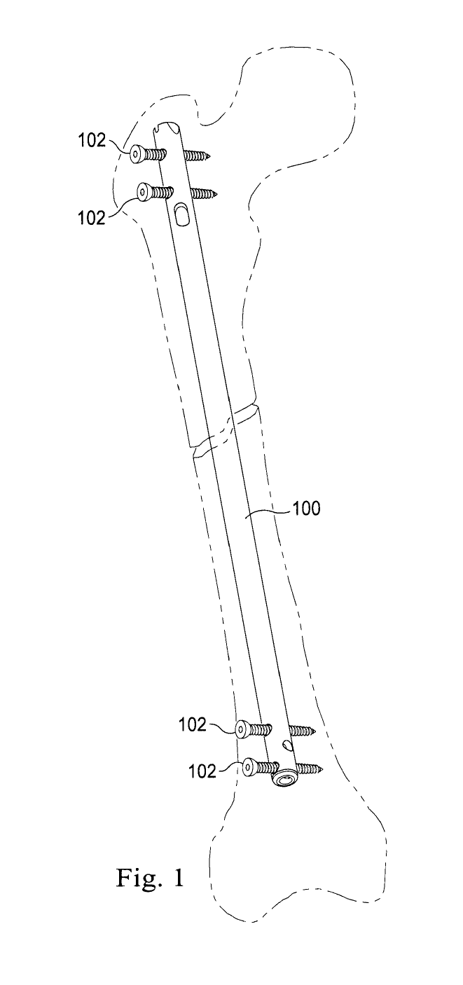

FIG. 1 illustrates a femur of a patient divided into a proximal bone segment and a distal bone segment. An exemplary intramedullary nail 100 extends along the intramedulally canal of the bone segments. As can be seen, the nail 100 is anchored with interlocking screws 102 in both the proximal bone segment and the distal bone segment. However, because the intramedullary nail 100 often deflects when inserted into the bone segment, finding the distal holes for receiving the interlocking screws 102 in the intramedullary nail 100 can be challenging. The intramedullary nail implantation systems disclosed herein help identify the deformation of the intramedullary nail that occurs as a result of being passed through the intramedullary canal and then compensates for the deformation with a surgical guide in order to provide a more reliable method to distally lock the intramedullary nail.

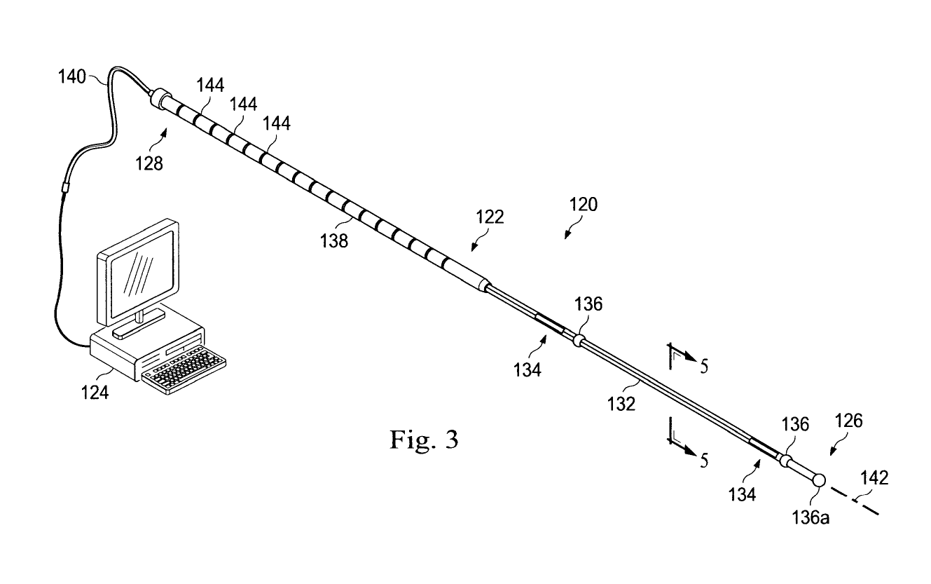

The intramedullary nail implantation systems disclosed herein include the intramedullary nail 100, a probe gage assembly (shown in FIG. 3), and jigs and drill guides as discussed herein that may be used to align one or more interlocking screws 102 with interlock holes in the intramedullary nail 100 when the intramedullary nail is disposed within a patient.

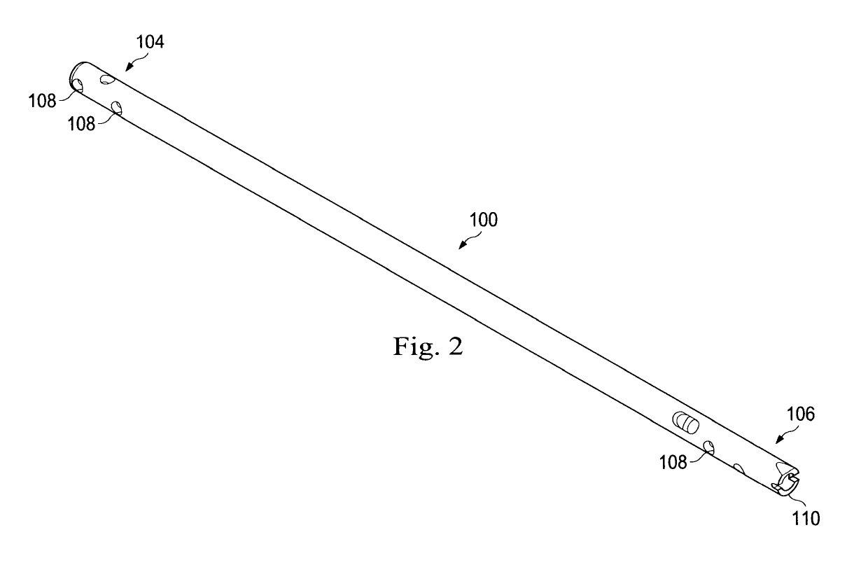

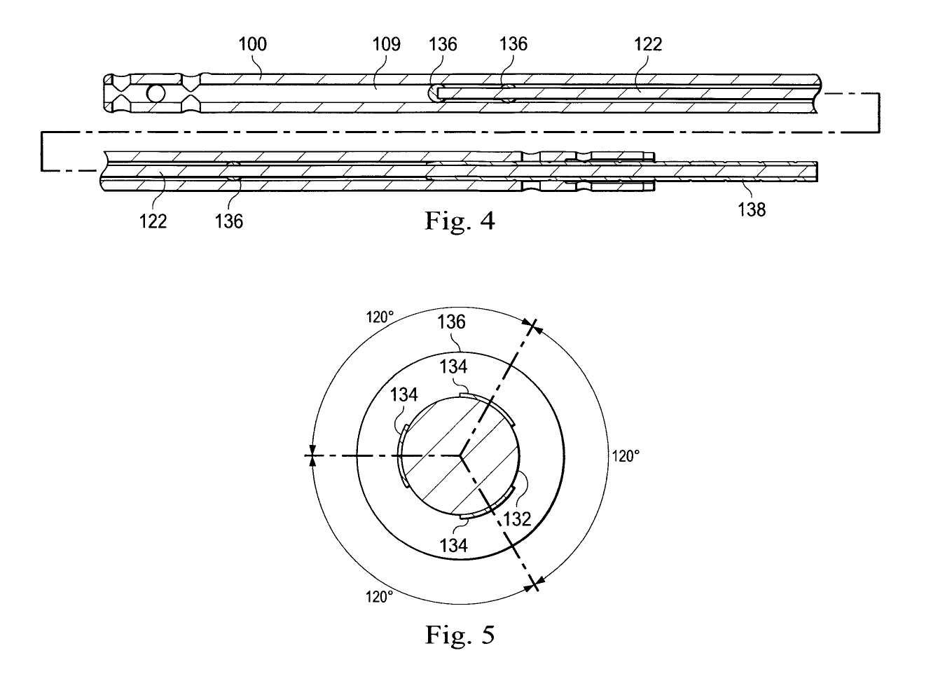

FIG. 2 shows the intramedullary nail 100 independent of the patient. The nail 100 includes a distal end 104, a proximal end 106, and includes interlock holes 108 arranged to receive the interlocking screws 102 shown in FIG. 1. In this embodiment, the nail 100 also includes an adapter interface 110 at the proximal end 106 shaped and configured to align with and connect to an adapter linked to a drill guide during use. The intramedullary nail 100 includes a canal 109 (FIG. 4) extending from the distal end 104 to the proximal end 106. This will be described further below.

FIG. 3 shows a probe gage assembly 120 that may be used to determine the location of the interlock holes 108 in the nail 100 when the nail is implanted within a patient. The assembly 120 includes a probe 122 and a processing system 124. The probe 122 is configured to fit within the canal 109 of the intramedullary nail 100 as shown in FIG. 4. FIG. 4 is a cross-sectional view of a portion of the probe 122 disposed within the canal of the intramedullary nail 100.

Still referring to FIG. 3, the probe 122 extends between a distal portion 126 and a proximal portion 128. It also includes a main core 132, sensing devices 134, centering elements 136, a sleeve 138, and a communication element shown as a wire 140. The wire 140 connects with the processing system 124 and is configured to carry data or other signals for processing by the processing system 124.

The main core 132 is a flexible member that runs the length of the probe 122 from the distal portion 126 to the proximal portion 128 and serves to provide the necessary structural integrity and flexibility to negotiate the canal 109 within the intramedullary nail 100. This main core 132 has a longitudinal axis 142 and can be made from a variety of materials such as high strength metal wire or composite materials. Although many different types of materials may be used, the material of the main core 132 is selected to have sufficient strength and flexibility to be able to, without permanent upset, negotiate the bends in the canal of the intramedullary nail 100 when the intramedullary nail 100 is implanted into the intramedullary canal of a bone of a patient. In the embodiment shown, the cross-sectional shape of the main core 132 is circular. However, other cross-sectional shapes are envisioned and would be dependent on the characteristics of the canal shape and the desired arrangement of the sensing devices 134 attached to the main core 132.

In this exemplary embodiment, the sensing devices 134 are bonded to the main core 132. Here, the sensing devices 134 are linear strain gages arranged to detect strain in the core 132, as the strain is indicative of the deflection of the core 132, which can be used to find the deflection of the nail 100. In the embodiment shown, the sensing devices 134 are arranged in a circular array of three about the axis 142 of the main core 132. This can be seen in FIG. 5, showing a cross-sectional view taken along lines 5-5 in FIG. 3. Although an array of three sensing devices is shown in FIG. 5, other arrangements and other numbers of devices are also contemplated as discussed below.

The sensing device configuration of a circular array can detect bi-planar strain by simply plotting the strain readings at each sensing device 134 using a cylindrical coordinate system centered and aligned with the tangent of the core axis 142. The three strain magnitudes when plotted in a cylindrical coordinate system yield enough information to describe a plane in that same coordinate system. In the trivial case where all of the strains are zero or the same value, the plane described is normal to the tangent of the core axis 142. When the values differ, the resultant plane will be at some angle with respect to the axis tangent, and the plane within which that angle lies will also be available to define the bi-planar components of that angle. The probe therefore can obtain a collection of points or readings representing radial deflection of the implant.

FIG. 3 shows multiple arrays of sensing devices 134. These multiple arrays of sensing devices 134 are displaced axially and serve to provide feedback along segments of the probe 122 in a piecewise manner. A simple arrangement uses one segment and only one array. More complicated devices may have multiple segments and arrays that may provide increased accuracy and granularity. The measured segments together may be used to determine a composite deformation of the probe 122.

The probe 122 uses the centering elements 136 to maintain a centered position within the canal of the intramedullary nail 100. This may also make the multiple segments and arrays distinct. Here, the centering elements 136 are spherical balls, and serve at least two purposes: the first is to act as demarcation elements that divide the probe 122 into sensing segments with sensing devices for each segment, and the second is to guide and center the probe 122 in the canal within the intramedullary nail 100. At the end of the probe 122 is an additional centering element 136a that helps make certain that the trajectory of the probe end is tangent to the distal end 104 of the canal within the intramedullary nail 100 (FIG. 2). This may allow for extrapolation to the more distal location where the interconnecting screw cross holes 108 are located. Alternatively, one could register the strains at multiple depths as the probe 122 is either inserted into or removed from the intramedullary nail 100. These multiple depth readings would result in a series of end deflections or points in space, the multiple points taken in pairs would allow for the determination of end curvature, a single pair would give the slope of the end, three points taken in pairs would provide two slopes offset by a depth distance that would provide end curvature.

To accommodate the wide variety of nail lengths, the sleeve 138 is provided along the proximal portion 128 of the probe 122. In the embodiment shown, this sleeve 138 has grooves 144 corresponding to the incremental length of a modular nail adapter that is chosen for the particular nail being used. The sleeve 138 acts to allow placement of the probe 122 to the proper depth such that the active portion of the probe 122 is always distal of the proximal end 106 of the nail 100 and the corresponding interlocking holes 108 used for proximal fixation.

The sleeve 138 also serves to align the proximal portion 128 of the probe 122 with the proximal end 106 of the nail 100 such that the constraint on the probe 122 is that of a cantilever. The main core 132 can be made to slide axially within this sleeve 138 to facilitate a multiple of measurements with a fixed proximal end condition if desired for reasons mentioned above. Using the sensing devices 134, strain can then be measured at some known location between the centering elements 136 for each of the segments through the use of the strain gage arrays. Based on known end conditions of those segments in terms of the constraints, cantilever, simple support, etc. as well as compatibility amongst the segments, the strain at the particular location can them be used to determine the slope along the segments. This may accomplished with the processing system 124 shown in FIG. 3.

The processing system 124 is a computer system including a processing unit containing a processor and a memory. An output device, such as a display and input devices, such as keyboards, scanners, and others, are in communication with the processing unit. Additional peripheral devices also may be present. The processor may for example be a microprocessor of a known type. The memory may, in some embodiments, collectively represent two or more different types of memory. For example, the memory may include a read only memory (ROM) that stores a program executed by the processor, as well as static data for the processor. In addition, the memory may include some random access memory (RAM) that is used by the processor to store data that changes dynamically during program execution. The processor and the memory could optionally be implemented as respective portions of a known device that is commonly referred to as a microcontroller. The memory may contain one or more executable programs to carry out the methods contained herein, including joining, separating, storing, and other actions including Boolean actions. Data may be communicated to the processing system 124 by any known method, including by direct communication, by storing and physically delivering, such as using a removable disc, removable drive, or other removable storage device, over e-mail, or using other known transfer systems over a network, such as a LAN or WAN, including over the internet or otherwise. Any data received at the processing system may be stored in the memory for processing and manipulation by the processor. In some embodiments, the memory is a storage database separate from the processor. Other systems also are contemplated.

The processing system 124 may be configured and arranged to receive information over the wire 140, or through wireless communication methods that represent information or signals from the sensing devices 134. Using this information, the processing system 124 may be configured to calculate and output values or data representing the position of the interlock holes 108 of the nail 102. As described below, a surgical guide such as a drill guide may be aligned with the interlock holes based on settings output from the processing system 124.

The processing system 124 may be used to determine the slope along the probe 122, or along segments of the probe 122 based on the known end conditions of the probe or segments of the probe in terms of the constraints, cantilever, simple support, etc. as well as compatibility amongst the segments. An integration of the slopes along the length of the segments allows for the calculation of deflection at the end of each segment. An integration of all of the segments therefore results in a known end deflection and end slope that can be used to represent the location of the interlock holes in the intramedullary nail 100. If the nail 100 continues further than the probe 122, a simple extrapolation of this end deflection and slope can be used to determine the end condition of the interlock holes. One skilled in the art can appreciate that the number of segments used along with the corresponding strain gage arrays can be anywhere from one to multiple and the number of strain gage elements within each strain gage array can also be anywhere from one to multiple. The more of each that are available would in general lead to a greater degree of precision. A final balance between the number of elements and the degree of precision will be based on the particular application.

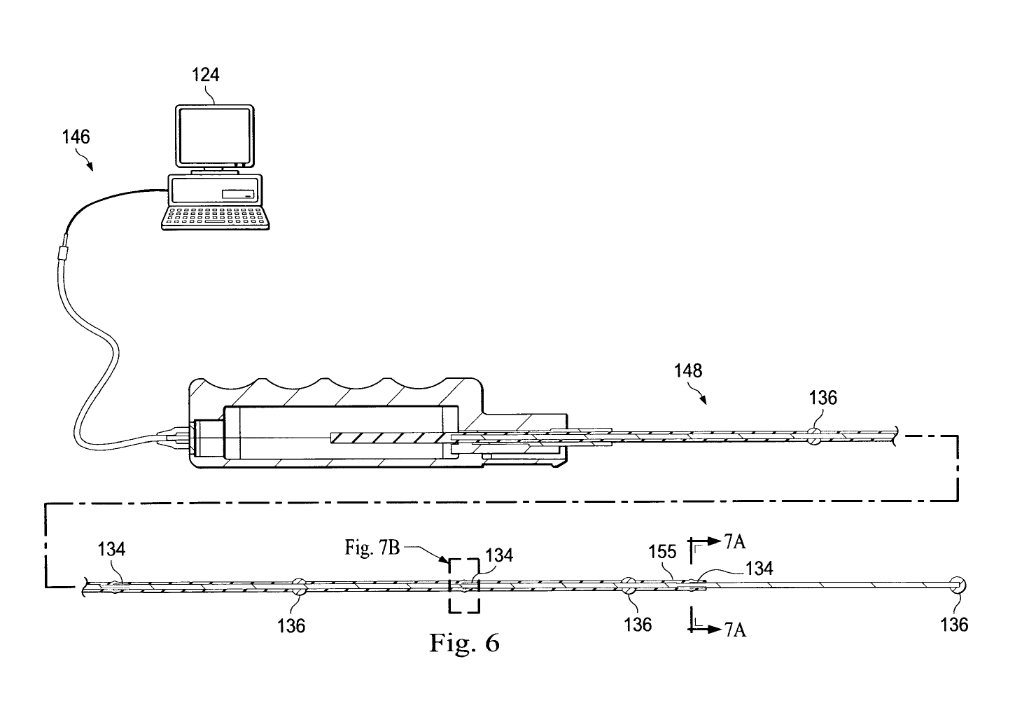

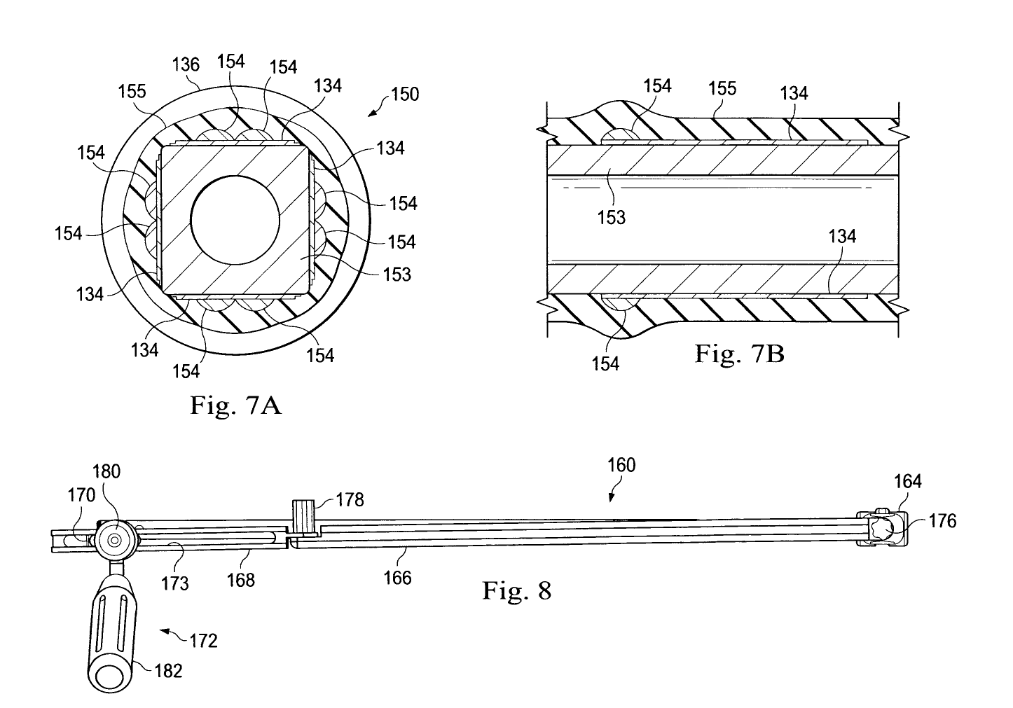

FIGS. 6, 7A, and 7B show another embodiment of a probe gage assembly 146 that may be used to identify the interlock holes 108 in the nail 100 when the nail is implanted within a patient. The assembly 146 includes a probe 148 and the processing system 124. Like the probe 122, the probe 148 is configured to fit within the canal 109 of the intramedullary nail 100.

The probe 148 includes a body 150, sensing devices 134, centering elements 136, a handle portion 152 and the communication element shown as the wire 140. The body 150 extends from the handle portion 152 and is configured to be introduced into the medullary nail as discussed above. In this embodiment, the body 150 can be selectively connected to or disconnected from the handle portion 152 as desired. Accordingly, the handle portion 152 may be used for any of multitude of probes having different sizes or characteristics. The body 150 may connect to the handle portion 152 using any known method, including, for example, snapping into the handle portion 152 using a compliant fastener and screwing into the handle portion 152. Of course other attachments systems are contemplated and in some embodiments, the body 150 is integral with the handle portion 152. The centering elements 136 are similar to those discussed above and their description will not be repeated here.

The body 150 is shown in cross-section in FIG. 7A and shown in detail in FIG. 7B. The body 150 includes a main core 153, solder connections 154, and a covering 155. In this embodiment, the main core 153 has a square cross-section. The solder connections 154 connect the sensing devices 134. The covering 155 may be a sheath such as shrink-tubing that protects the sensing devices 134 when the probe 148 is in use.

In FIG. 6, the probe 148 includes include three arrays of sensing devices 134. The sensing devices 134 are paired with opposing sides of the square main core 153 as shown in FIGS. 7A and 7B, allowing for two orthogonal pairs that then could be configured as two half bridges. The resulting strain imbalance for each pair then directly measures the strain in each of the corresponding planes. The advantage here is an increased sensitivity in the half bridge nature of the sensing device pairs and the direct measurement of the strains in each plane. This may come at the expense of an additional strain gage and more lead wires for each array. In the simplest case, a single strain gage could be used on the broad side of a flat rectangular core. This arrangement would only yield strain in one plane and therefore the probe would need to be rotated to at least one other location, e.g., orthogonal to the initial to register the strain in the other plane. A practical method for accomplishing this would be to rotate the probe 122 while recording the strain output and plotting this against a rotation index. This may be best accomplished using some form of rotational encoder to form the rotation index. A potential advantage of this approach would be that the rotation of the probe 122 would nullify any imbalance in the main core 132 caused by such things as gage to gage linearity, run out in the probe core, etc. This rotational approach could be used in probes having any number or arrangement of strain gages to the same effect.

In some instances, the distal end deflection and slope are determined using an algorithm stored and/or executed by the processing system 124. The output of the algorithm may include a series of adjustments that are intended to be used in the adjustment of surgical guide such as a jig 160 in FIGS. 8-10 that may form a portion of an intramedullary nail implantation system. This jig 160 has an ability to change the angle of a distal drill guide in two planes along with the ability to adjust the overall length between the proximal and distal end. The user would simply adjust the jig 160 to the output settings from the processing system 124 which would ensure that the drill guide and the distal holes were in alignment. In one embodiment, the probe 122 is left in place while adjustments are made. In another embodiment, the probe strain is stored in the processing system 124 and calculations to determine the jig settings would be made after the probe 122 is removed. This allows the user to lock the proximal end 106 of the nail 100 in place prior to locking the distal end 106. This gives the user additional flexibility regarding the reduction of the fracture and ensures that the proximal portion is properly positioned prior to the locking of the distal portion.

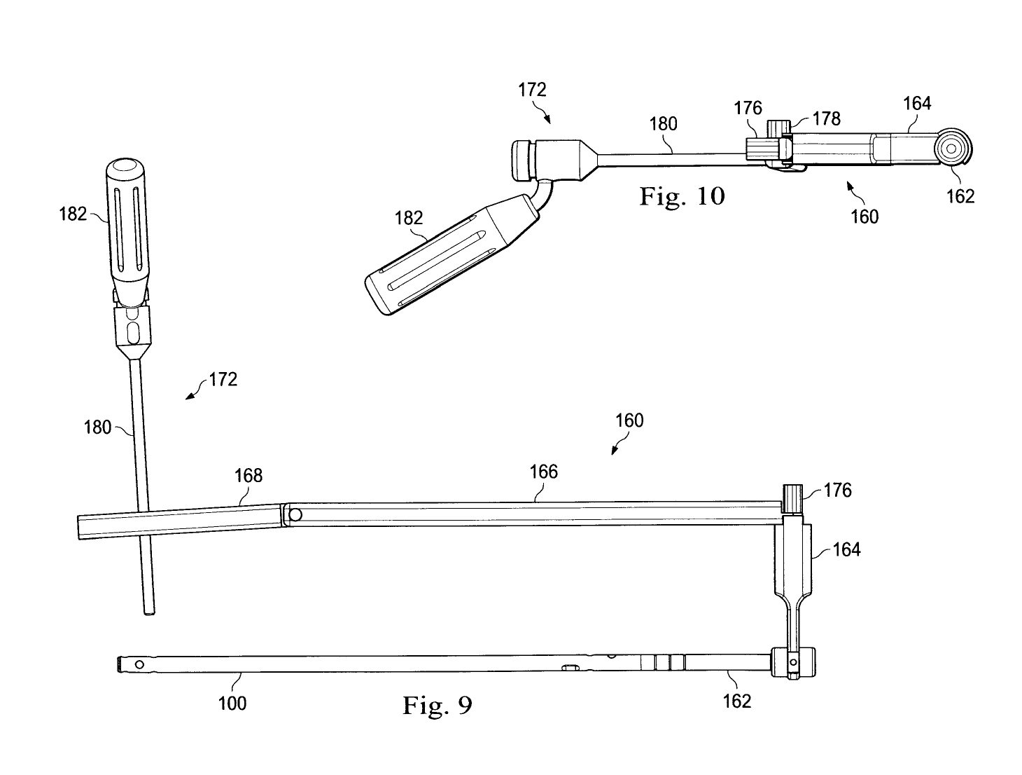

FIG. 8 shows a top view, FIG. 9 shows an elevation view, and FIG. 10 shows a side view of the jig 160 that may be adjusted based on outputs from the processing system 124 to align a drilling guide with the interlock holes of the intramedullary nail 100. Referring to FIGS. 8 and 9, the jig 160 includes a modular nail adapter 162, a cross member 164, a first arm 166, a second arm 168, a drill cartridge 170, and a drill guide 172. Adjustment knobs 176 and 178 are used to change the relative angles at joints or pivot points between the first arm 166, the second arm 168, and between the cross member 164 and the first arm 166. The settings output from the processing system 124 may be the settings on the knobs that align the drill guide with the interlock holes 108 in the intramedullary nail 100. Accordingly, by merely setting the jig 160 at the output settings, a surgeon can drill holes for the interlocking screws.

As best seen in FIG. 9, the jig 160 connects to the intramedullary nail 100. The modular nail adapter 162 fits over the sleeve 138 of the probe 122 (shown in FIG. 3), which is disposed within the canal of the intramedullary nail 100. In addition, the modular nail adapter 162 is configured to engage the adapter interface 110 of the intramedullary nail 100 and align itself coaxially with the intramedullary nail 100. In this example, the adapter interface 110 is configured to cooperate with the modular nail adapter 162 to prevent relative rotation between the intramedullary nail 100 and the modular nail adapter 162. Accordingly, the modular nail adapter 162 is rotationally fixed relative to the intramedullary nail 100.

The cross member 164 connects to and extends laterally from the modular nail adapter 162. The first arm 166 connects to the cross member 164 and extends at a transverse angle from the cross member 164. Accordingly, the first arm 166 may lie within a plane generally parallel to a plane containing the intramedullary nail 100. The first arm 166 is pivotally connected to the cross member 164 and is configured to be rotated about an axis through the cross member 164 by rotating the knob 176. The knob may be configured with detents that provide incremental rotation. The knob 176 may also be arranged to move relative to particular settings that may be displayed on the knob, the arm 166, the cross member 164 or otherwise so that a surgeon may rotate the knob 176 to a particular setting that may be output from the processing system 124 based on the detected position of the nail.

The first arm 166 and the second arm 168 are rigidly extending elements connected at a pivot joint at the adjustment knob 178. The adjustment knob 178 may control the amount of rotation of the second arm 168 relative to the first arm 166. In a manner similar to the knob 176, the knob 178 may also be arranged to move relative to particular settings that may be displayed on the knob, the arm 166, the arm 168 or otherwise so that a surgeon may rotate the knob 17 to a particular setting that may be output from the processing system 124 based on the detected position of the nail.

The second arm 168, at least in the embodiment shown, is configured with a sliding slot 173 (FIG. 8) formed therein that accommodates the drill cartridge 170. Accordingly, the drill cartridge 170 may slide within the slot 173 in the axial direction of the second arm 168 so that the drill cartridge 170 may align with the interlock holes in the intramedullary nail 100.

The drill cartridge 170 is configured to carry the drill guide 172. Accordingly, as the drill cartridge 170 moves along the axis of the second arm 168, the drill guide 172 also moves along the second arm 168. The drill guide 172 includes a drill guide body 180 and a handle 182. The drill guide body 180 extends through the drill cartridge 170 and in this case, through the second arm 168 and is a guide for the actual drill instrument when the jig 160 is aligned with the interlock holes in the intramedullary nail 100.

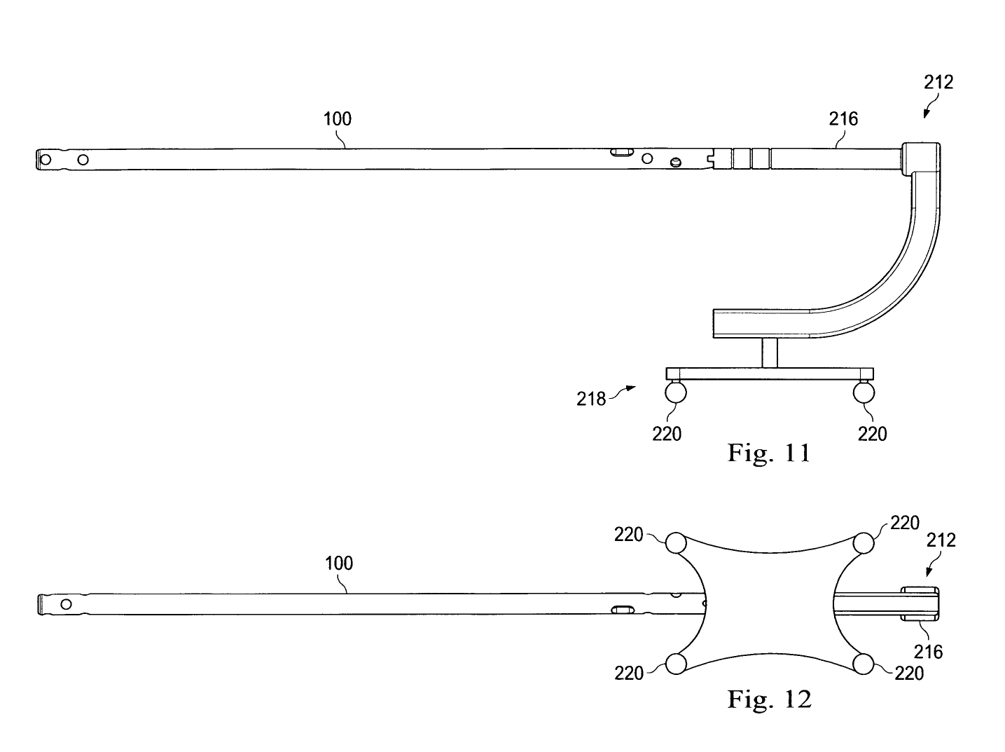

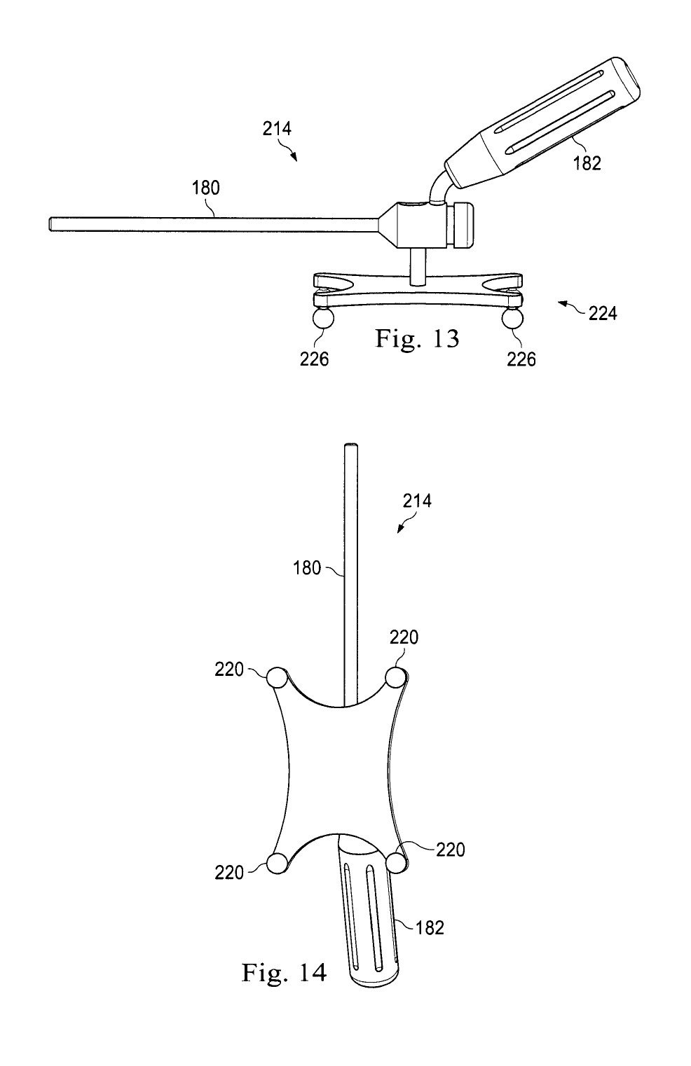

FIGS. 11-14 show another portion of an intramedullary nail implantation system as a jig that may be used with the probe assembly 120 to determine the location of the interlock holes in the intramedullary nail 100 and to align a drill guide with the interlock holes. Here, the intramedullary nail implantation system includes the intramedullary nail 100, the probe assembly 120 with a modified processing system 124, and a jig that includes a nail insertion jig 212 and a free floating drill guide 214. The intramedullary nail implantation system in this embodiment is a computer assisted surgery (CAS) system. The nail insertion jig 212 is shown in FIGS. 11-12 and the drill guide 214 is shown in FIGS. 13 and 14.

In this embodiment, the nail insertion jig 212 includes a modular nail adapter 216 and a tracking marker 218 fixedly connected thereto. The modular nail adapter 216 is similar to the modular nail adapter 216 and is configured to connect to the intramedullary nail 100 as it is disposed in the patient. The tracking marker 218 extends from the adapter 216 and includes an array of targeting spheres or other geometrical features 220 (based on the CAS system being used) attached to the proximal end of the nail insertion jig 212. The geometrical features 220 in this embodiment comprise a set of four spheres. However, other arrangements or other numbers of spheres are contemplated.

The free floating drill guide 214 also includes the drill guide body 180 and the handle 182 as discussed above with reference to FIGS. 8-10. In addition, it includes a tracking marker 224 fixedly connected thereto that includes an array of targeting spheres or other geometrical features 226 (based on the CAS system being used) attached to the proximal drill guide body 180. Again, in this example, the geometrical features 226 comprise a set of four spheres.

The processing system 124 in this embodiment still receives information from the probe 122 as discussed above to determine the deflection of the nail 100 in order to identify the location of the interlock holes 108. However, the processing system also includes a camera system configured to illuminate and detect the geometrical features 220, 226 on the nail insertion jig 212 and the drill guide 214. By identifying the location of the geometrical features 220, 226 in the nail insertion jig 212 and the drill guide 214, the system may be configured to determine the relative locations of the nail insertion jig 212 and the drill guide 214. Taking into account the deflection of the intramedullary nail 100 as determined by the probe 122, the location of the interlock holes can also be determined when the nail is disposed within the patient.

The processing system 124 may do this by creating a reference coordinate system. Based on the fixed array, a model of the nail insertion jig 212 and intramedullary nail 100 being used would be placed in that coordinate system. With the probe 122 inserted into intramedullary nail 100 and the intramedullary nail 100 being inserted into the patient, strain readings are taken as described above and these readings are used to alter the model of the intramedullary nail 100 such that the nail model would match the deflected model as registered by the inserted probe 122. The processing system 124 then presents this information graphically to the surgeon in accordance with the methods of the particular CAS system such that the free floating drill guide 214 could be positioned in a state of alignment with the various interlock holes 108 used to receive the interlocking screws and lock the nail.

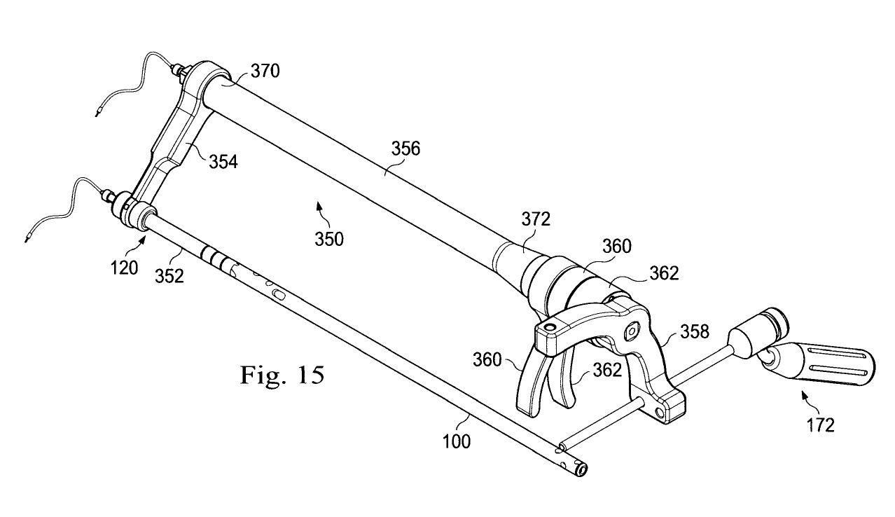

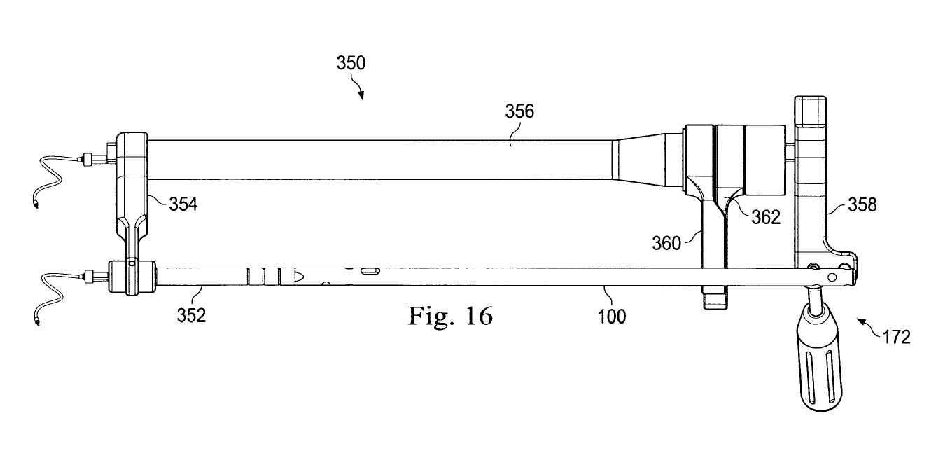

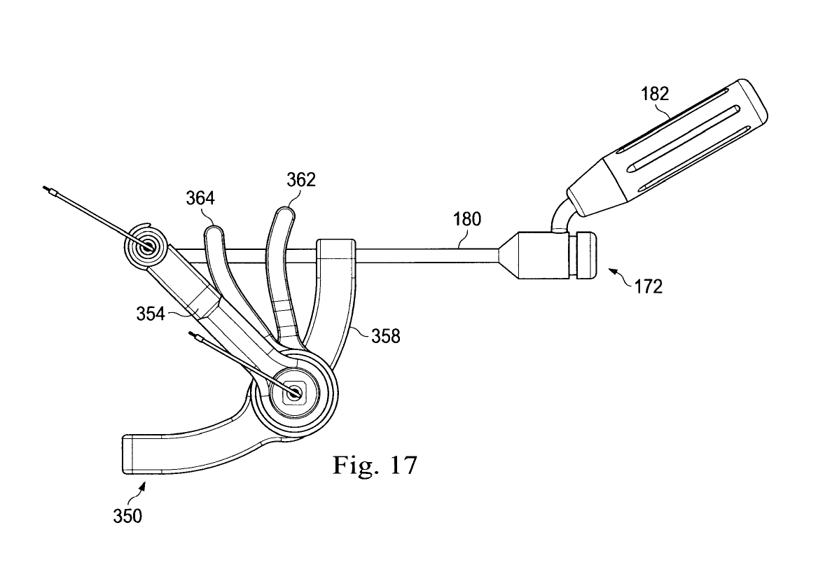

Yet another embodiment of a portion of an intramedullary nail implantation system is shown in FIGS. 15-23. Here, the intramedullary nail implantation system includes the intramedullary nail 100, the probe assembly 120, and another insertion jig 350. The intramedullary nail 100 is again attached to the insertion jig 100, which is then inserted into the patient. The insertion jig 350 in this embodiment includes a nail analog used to mimic the deflection of the actual nail to align a drill guide with the actual intramedullary nail 100. This nail analog is flexible and has the same or similar inner canal geometry as does the nail such that the bending characteristics are similar. The purpose of this nail analog is to allow the surgeon to manipulate this analog until it matches the deformation of the nail in use. To accomplish this, the probe 122 is first inserted into the nail 100, strain readings are taken and this information is stored. The probe 122 is then removed from the nail 100 and inserted into the nail analog. The jig 350 is manipulated until the readings received from the nail probe 122 within the nail analog match those readings that were stored when the probe 122 was inserted in the nail 100. In an alternative embodiment the jig 350 might use a separate probe 122 installed in the nail analog with one installed in the nail 100 itself. This would eliminate the need for data storage and could in principle allow for a simple analog balancing between the two probes negating the need for any computational capacity. In still another embodiment the nail analog and/or the nail 100 could have the strain gages permanently installed negating the need for a separate probe.

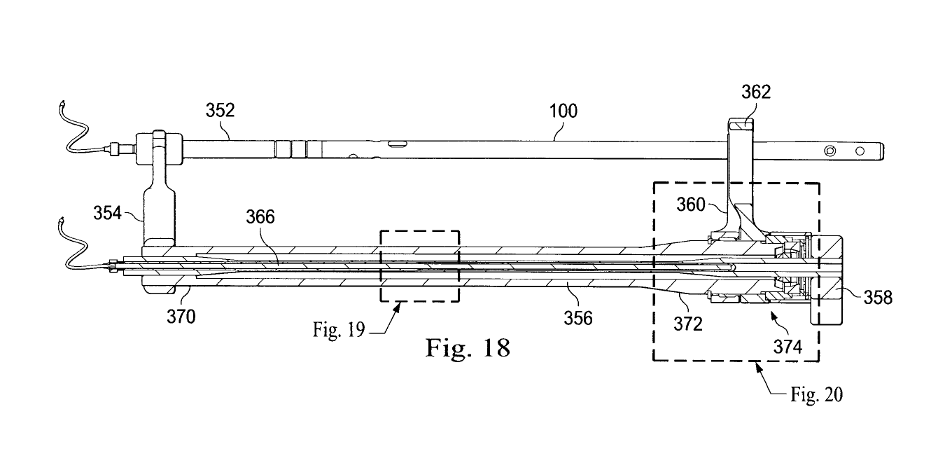

The insertion jig 350 with the nail analog is discussed in greater detail below. The jig 350 includes a modular nail adapter 352, a cross member 354, a rigid tubular body 356 containing the nail analog 366 (FIG. 18), a drill guide targeting device 358, the drill guide 172, and a pair of gripping handles 360 and 362.

The modular nail adapter 352 is similar to the modular nail adapter 216 and is configured to connect to the intramedullary nail 100 as it is disposed in the patient. The cross member 354 extends from the modular nail adapter 352 and rigidly connects the intramedullary nail 100 to the tubular body 356 containing the nail analog 366. The tubular body 356 has a proximal end 370 and a distal end 372 and extends from the cross member 354 toward the drill guide 172, and abuts the gripping handles 360, 362. The gripping handles 360, 362 are used to manipulate the nail analog 366 in a manner to mimic the strain on the actual intramedullary nail 100, so that the drill guide disposed at the end of the nail analog is deflected to correspond with the deflection of the intramedullary nail 100.

As indicated above, the rigid tubular body 356 holds the nail analog 366. This is best seen in FIG. 18. At the proximal end 370 of the rigid tubular body 356, the nail analog 366 is held fixed with some allowance for axial movement, while at the distal end 372 of the rigid tubular body 356, the nail analog 366 is supported within a manipulation assembly 374 that allows and causes the distal end of the nail analog 166 to move radially, axially and to some degree, to change its end trajectory from a coaxial condition to a deflected condition that matches the deflected condition of the intramedullary nail 100. In one exemplary aspect, the manipulation assembly 374 deflects the nail analog to change its end trajectory to a condition where if the end trajectory was revolved around the axis of the rigid tubular body 356, the resulting form would be that of truncated conical form. This truncated conical form is the working envelope within which all positions and poses of the drill guide targeting device 358 can be placed. While the gripping handles 360, 362 are squeezed together, the nail analog 166 and thus the drill guide targeting device 358 is free to be manipulated within that envelope. Once the gripping handles 360, 362 are released, drill guide targeting device 358 is locked in its last position within that working envelope, allowing the surgeon to then insert the drill guide 172 within the targeting device 358 such that an intersection of a drilling instrument in the drill guide 172 and the interlock holes 108 can be assured.

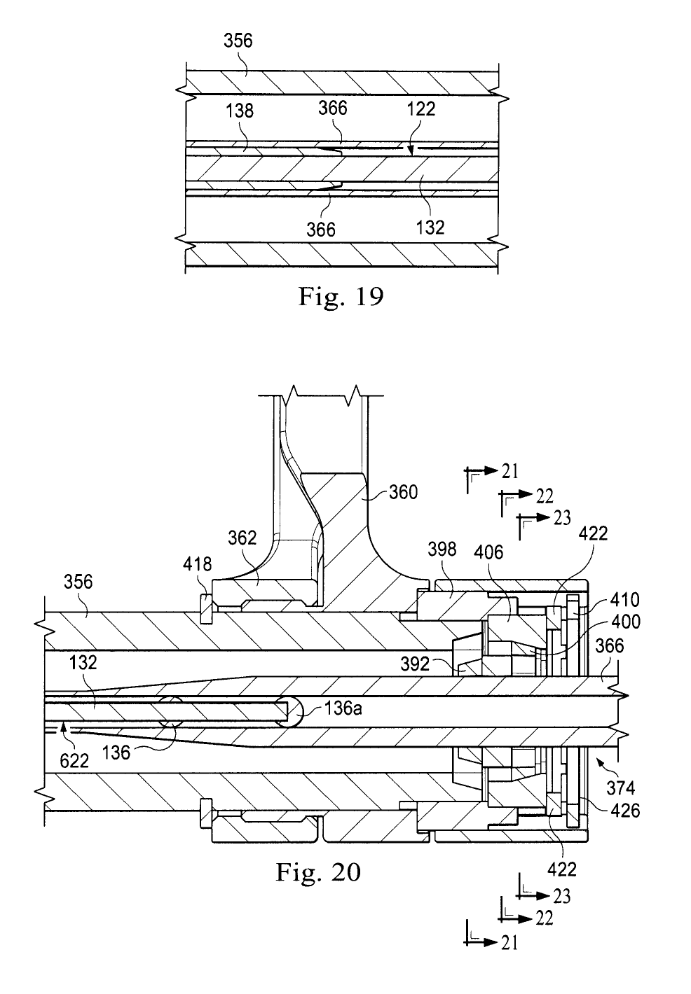

FIG. 19 is a blown-up view of a portion of FIG. 18. It includes a portion of the nail analog 366 and a portion of a probe 120, having the main core 132, the sleeve 138, and other elements of the probe 122. As previously indicated, the system may include two probes 122, or may include one probe 122 that is first introduced to the intramedullary nail 100 and then later introduced to the nail analog 366.

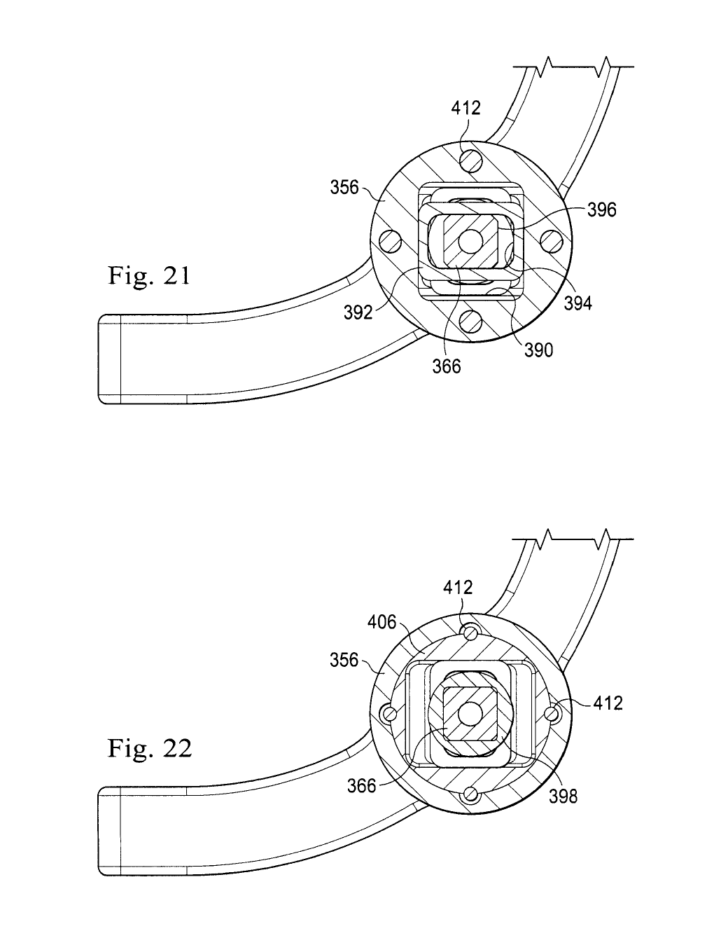

The manipulation assembly 374 is described with reference to FIGS. 20-23. The manipulation assembly 374 is also arranged to lock and unlock the working envelope in the manner discussed above. The locking and unlocking of the working envelope is accomplished using the mechanism shown in FIG. 20. In part, the manipulation assembly 374 includes what may be referred to as a stacked double Oldham coupling. Referring to FIG. 21, taken through lines 21-21 in FIG. 20, the Oldham coupling includes a first rectangular cavity 390, having a rectangular form integral within the rigid tubular body 356. Within that cavity 390 sits a rectangular plate 392. One of the sides of the rectangular plate 392 has the same dimension as one side of the rectangular cavity 390, and the other side of the rectangular plate 392 has a dimension smaller than the other side of the rectangular cavity 390. This rectangular plate 392 is mated to the rectangular cavity 390 such that it is free to slide along the plane of the like dimensioned sides to the extent defined by the clearance between the other unlike dimensioned sides. That is, the rectangular plate 392 can move in one direction relative to the rectangular opening 390.

The rectangular plate 392 also has a rectangular opening 394 within it that mates with a rectangular section 396 or tongue of the nail analog 366 along one side, but not the other. That is, the rectangular section 396 of the nail analog 366 has a dimension that is the same as one side of the rectangular opening 394, while the other side of the rectangular opening 394 is larger in dimension than the other side of the rectangular section 396 of the nail analog 366. This permits the rectangular section 396 of the nail analog 366 to move in one direction relative to the rectangular opening 394. The orientation of this side having the clearance is orthogonal to the orientation of the side with clearance in the rectangular plate 392 and rectangular opening 390.

Referring to FIG. 22, taken along lines 22-22 in FIG. 20, a spacer bushing 398 is disposed axially along the nail analog 366. The spacer bushing 398 has a face mated to the previously described rectangular plate 392.

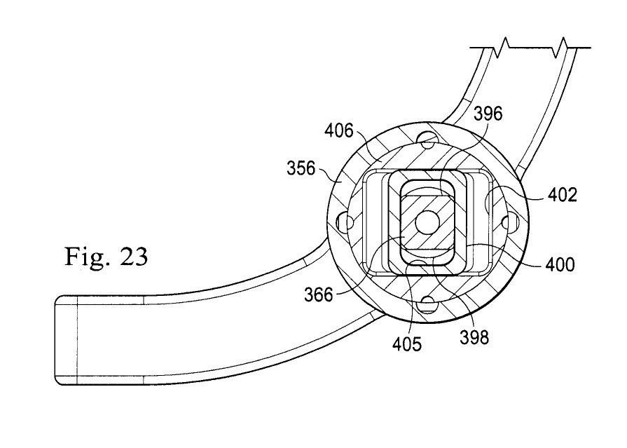

Referring to FIG. 23, taken along lines 23-23 in FIG. 20, another rectangular plate 400 is disposed on top of the spacer bushing 398. This is axially disposed along the nail analog 366, and is held with a rectangular cavity 402, in an arrangement similar to the previous plate 392 in a rectangular cavity 390 discussed above. The nail analog 366 is disposed to slide in one direction in a rectangular cavity 405. However here, the mated relationship between the plate 400 and the rectangular cavity 402 and the rectangular cavity 405 and the nail analog 366 is orthogonal to the previous description. The resulting arrangement allows the rectangular section 396 or tongue of the nail analog 366 to move radially with respect to the rigid tubular body 356. The fact that the rectangular plates 392, 400 are disposed to be offset axially along the nail analog 366 also allows for a change in trajectory given that the centers of the two Oldham coupling arrangements can be non-coincident with respect to each other in a coordinate system aligned with the center axis of the rigid tubular body 356.

A support bushing 406 in FIGS. 22 and 23 is free to slide axially within the rigid tubular body 356 and is spring loaded against the stack of Oldham couplings with a plate spring 410 shown in FIG. 20. In this embodiment the mated sides of the Oldham couplings are tapered as shown in FIG. 20, such that an axial clamping force generated by the plate spring 410 forces the tapered geometry into a state of interference effectively removing any lash that would be present. The support bushing 406 is held from rotation within the rigid tubular body 356 through the use of four cylindrical pins 412 in FIGS. 21 and 22. These pins 412 also bear against the bottom face of the support bushing 406 and the top face of gripping handle 360 in FIG. 20. The gripping handle 360 is threaded into the gripping handle 362 in FIG. 20. Rotation of the gripping handle 360 relative to gripping handle 362 either causes them to move further apart or closer together. In moving them further apart, the proximal face of the gripping handle 362 bears against a snap ring 418 in FIG. 20, forcing the opposite face of the gripping handle 360 against the four cylindrical pins 412 which further bear against the support bushing 406 in FIGS. 22 and 23, supporting the spring load that is applied to the support bushing 406 by a ring 422 in FIG. 20. This support of the spring load releases the clamp load on the rectangular plates 392, 400, thereby freeing up the mechanism, allowing manipulation of the nail analog 366. Rotating the gripping handle 360 relative to gripping handle 362 such that they move closer together once again allows the spring force to bear on the rectangular plates 392, 400 locking the mechanism and the nail analog 366. A biasing spring (not shown) could be used between the gripping handles 360 and 362 to either rotate them apart or together depending on whether the surgeon desires the normal or neutral state to be either locked or free. The entire assembly is held within the rigid tubular body 352 using a snap ring 426 in FIG. 20.

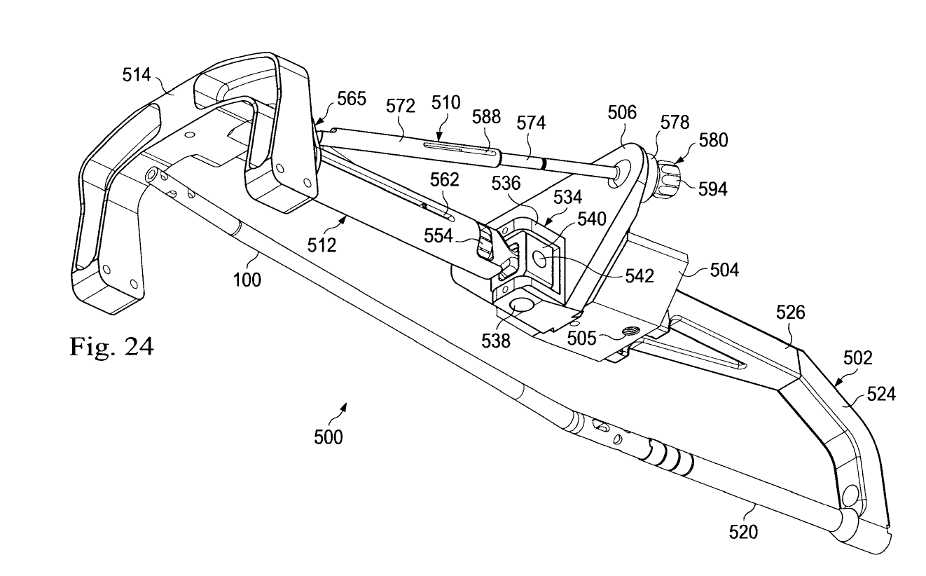

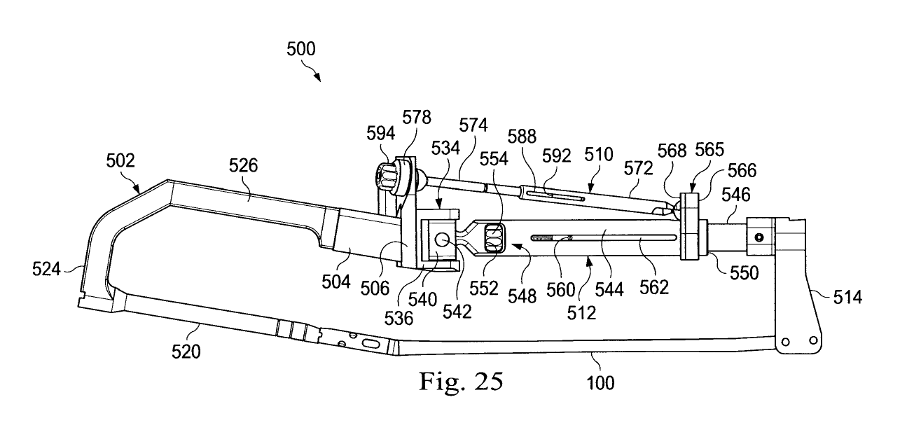

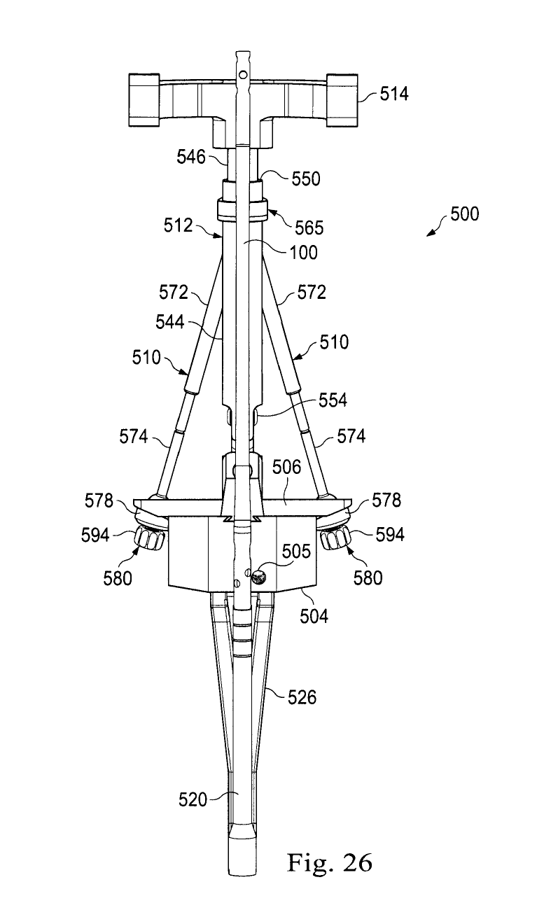

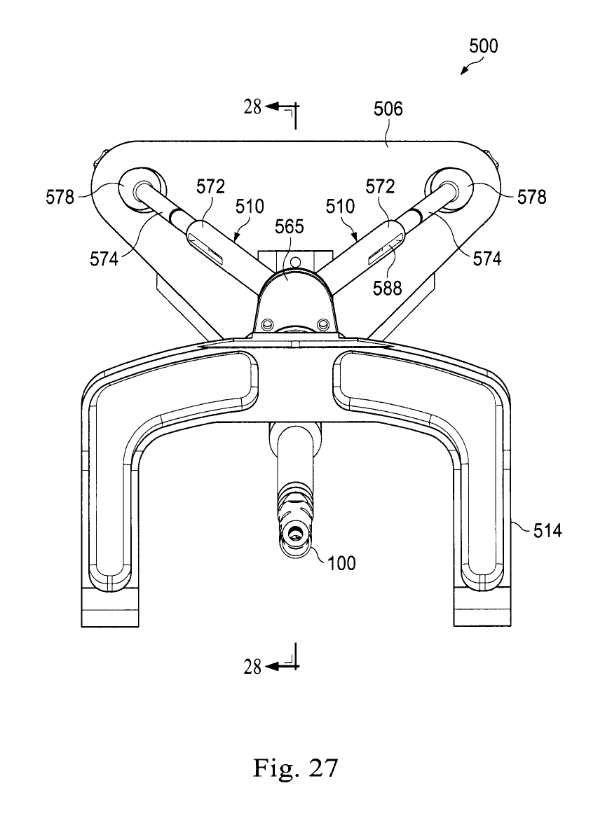

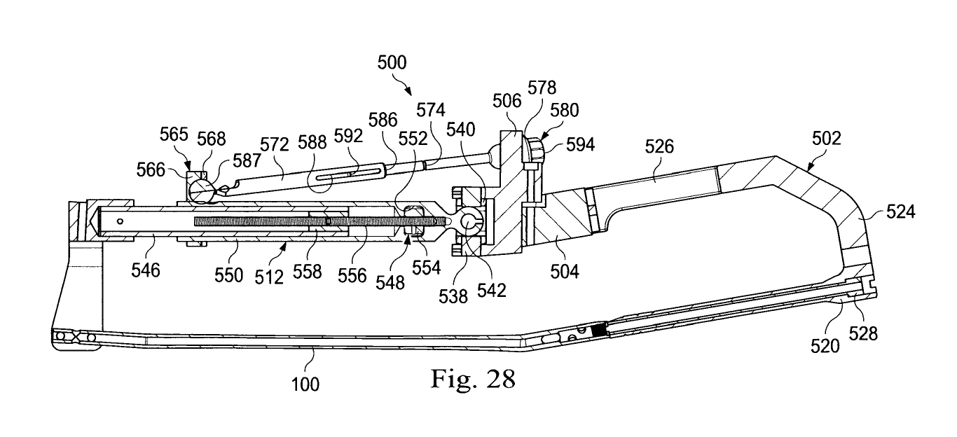

Yet another embodiment of a portion of an intramedullary nail implantation system is shown in FIGS. 24-28. Here, the intramedullary nail implantation system includes the intramedullary nail 100, the probe assembly 120, and another insertion jig 500. Like the embodiments previously described, the particular jig 500 can be manipulated in three degrees of freedom. However, the jig 500 can be positively driven in each of those degrees while some other embodiments herein are simply loosened and placed into a particular orientation. Such a drivable design may make adjustments easier and more accurate for the surgeon.

The intramedullary nail 100 is again attached to the insertion jig 500, which is then inserted into the patient. The insertion jig 500 in this embodiment includes a modular nail adapter 502, a proximal block 504, a base 506 connected to the proximal block 504, two adjustment struts 510, an adjustable base strut 512, and a distal drop 514 that may form or may support the drill guide.

The modular nail adapter 502 is arranged to interface with an adapter interface 110 at the proximal end 106 of the intramedullary nail 100 in the manner discussed above. In this embodiment, the modular nail adapter 502 is a U-shaped element having a rigid portion 520 arranged to extend from the intramedullary nail 100, a guide handle portion 524, and a block connector 526 configured to connect the proximal block 504. The rigid portion 520 includes a guide bolt 528 therein configured to screw into an end of the intramedullary nail 100 at the interface 110. Accordingly, in this embodiment, the interface includes a threaded connection to the guide bolt 528. In addition, the guide bolt 528 is hollow to receive the probe 122 (FIG. 3). The block connector 526 splits into two angled arms providing rigid stability to the proximal block and helping strengthen the overall jig 500.

The proximal block 504 is attached to the modular nail adapter 502 and serves as a stable anchor for other elements of the jig 500. In some exemplary embodiments, the proximal block 504 includes guide holes 505 (FIG. 24) that align with one or more of the proximal interlock holes 108 in the intramedullary nail. Since deflection of the intramedullary nail 100 is minimal at the proximal end, the proximal block 504 may not need to be adjusted to maintain alignment with the proximal interlock holes 108. The proximal block 504 may also come in different varieties to accommodate different nail systems and allow for the connection of different jig configurations.

The base 506 is attached to the proximal block 504 and serves as an immovable reference off of which all movements are based. That is, the proximal block 504 forms a reference point for the two adjustment struts 510 and the adjustable base strut 512. It is configured to anchor proximal ends of the two adjustment struts 510 and the adjustable base strut 512. In this embodiment, the base 506 includes spherical seat portions 530 for the proximal ends of each of the two adjustment struts 510. Accordingly, the two adjustment struts 510 are able to pivot about a spherical rotation point. In the embodiment shown, these spherical seat portions 530 are formed as concave surfaces formed in a back side of the base 506.

The base 506 also includes a seat 534 for the adjustable base strut 512. The seat 534 is a universal joint formed with a bracket 536, a first pivot pin 538, a revolute block 540, and a second pivot pin 542. The first pivot pin 538 extends between arms or sides of the bracket 536, and the revolute block 540 pivots or rotates about the axis of the first pivot pin 538. In some embodiments, for convenience, the pivot pin 538 is actually comprised of two pivot pins connected to the revolute block 540 and the two pivot pins do not pass all the way through the revolute block 540. This maintains the space in the revolute block 540 for additional components of the seat 534. The second pivot pin 542 extends between arms or sides of the revolute block 540 and passes through a hole in the base strut 512. Accordingly, the seat 534 on the base 506 allows the base strut 512 to pivot about the axis of the second pivot pin 542. As such, the base strut 512 can elevate relative to and rotate about a plane that is at some known orientation to the intramedullary nail 100 providing two degrees of freedom.

The base strut 512 extends from the seat 534 on the base 506 and includes a base portion 544, an extension portion 546, and an adjustment portion 548. The base portion 544 connects to the second pivot pin 542 and is therefore anchored to the base 506. In this embodiment, the base portion 544 includes a distal open end 550 and includes a knob seat 552. The extension portion 546 is slidably engaged and fixed in rotation relative to the base portion 544 through the use of an extension bushing 558 and an indicator pin 560. As such, the extension portion 546 is in a telescoping relationship with the base portion 544 and allows the base strut 512 to be extended along an axis in the axial direction. The adjustment portion 548 includes a knob 554, a threaded extension screw 556, and an extension bushing 558. The knob 554 is disposed in the knob seat 552 and can be accessed by a user to rotate about the threaded extension screw 556. The knob 554 is fixed to the threaded extension screw such that rotation of the knob 554 turns the extension screw 556. The extension screw 556 is threadably connected to the bushing 558. Because of the threaded connection, when the extension screw 556 rotates, the bushing 558 moves along the axis of the extension screw 556 and along the axis of the base strut 512. The bushing 558 is fixedly connected to the extension strut 546. Accordingly, when the bushing 558 moves, the extension strut 546 also moves axially, increasing the length of the base strut 512 and providing a third degree of freedom. The indicator pin 560 connected to either the extension strut 546 or the bushing 558 slides within a slot 562 on the base portion 544. In some embodiments, the base portion 544 includes indicia along the slot 562 representative of a setting or length of the base strut 512. A user may observe the location of the indicator pin 562 relative to the indicia to track the settings of the base strut 512.

A distal anchor 565 formed of a base plate 566 and a top plate 568 is also connected to the base portion 544 of the base strut 512. This anchor 565 connects the distal ends of the two adjustment struts 510 to the base strut 512. The base plate 560 includes a spherical seat for receiving spherical portions of the distal ends of the adjustment struts 510. The top plate 562 is used to maintain the distal ends of the adjustment struts 510 in the seat in the base plate 560.

The two adjustment struts 510 control the elevation and rotation of the base strut 512. Each of the adjustment struts 510 includes an adjustment strut base 572, a strut screw 574, a spherical bushing 578, and an adjustment portion 580. The adjustment strut base 572 includes a proximal open end 586, a semicircular distal end 587 opposite the open end 586, and a slot 588. The adjustment strut base 572 is in a telescoping relationship with the strut screw 574 and allows the adjustment strut 510 to be extended along an axis in the axial direction. That is the strut screw 574 is disposed within the open end 586 of the adjustment strut base 572. The semicircular distal end 587 forms one-half of a sphere and cooperates with a semicircular distal end of the other adjustment strut to form a common spherical joint to seat within and pivot relative to the distal anchor 565. The slot 588 extends axially along the adjustment strut base 572 and, like the slot 562 discussed above, includes indicia representing a length or position of the adjustment struts 510.

The strut screw 574 threadably attaches to the adjustment strut base 572 and may be axially displaced by rotation of the strut screw 574. An indicator pin 592 disposed relative to the strut screw 574 moves with the strut screw 574 in the slot 588, and the user may observe the location of the indicator pin 592 relative to the indicia to track the settings of the adjustment strut.

The strut screw 574 is individually connected by spherical joints to the base 506 through the spherical bushing 578 in the base 506. The spherical bushing 578 is disposed on the strut screw 574 and allows the strut screw 574 to pivot relative to the spherical seat portions 530.

The adjustment portion 580 includes an adjustment knob 594 that may be rotated to turn the strut screw 574 within the adjustment strut base 572 to displace the adjustment strut base 572 to either increase or decrease the length of the adjustment strut 510. The adjustment portion 580 may also be considered to include the threaded portions of the adjustment strut base 572 and the strut screw 574. The action of the adjustment struts 510 together results in the base strut 512 either elevating, rotating, or some combination of the two relative to the base 506 and, thus, relative to the intramedullary nail 100.

The distal drop 514 forms a drill guide or receives a drill guide or other surgical instrument configured to align with the interlock holes in the intramedullary nail 100. It is connected to the distal end of the base strut 512 and is configured to move in any direction by manipulation of the adjustment portions 548 and 580. Here, it is C-shaped and extends to both sides of the intramedullary nail 100. The settings output from the processing system 124 may be the settings on the knobs that align the drill guide with the interlock holes 108 in the intramedullary nail 100. Accordingly, by merely setting the jig 500 at the output settings, a surgeon can drill holes for the interlocking screws.

Operation of the jig 500 again is based upon the output of the probe 122 and the computations performed within the processing unit 124 the output of which would direct the surgeon to make the appropriate adjustments to the adjustment knobs 554, 594 such that the holes contained within the distal drop 514 are maintained in alignment with the interlock holes 108 in the intramedullary nail 100, even when the intramedullary nail 100 is deflected.

Each of the aforementioned embodiments rely on a relatively consistent geometric relationship between the nail analog and the nail in use as well as a consistent performance regarding the sensor probe 122. In order to overcome variance that may be introduced by manufacturing tolerances, the rigidity of the component parts, or other factors, a calibration process may be undertaken between the intramedullary nail 100 and any jig prior to insertion into the body. To do this, with the intramedullary nail 100 attached to the jig, and both the jig and intramedullary nail 100 in a free state, the intramedullary nail 100 is deflected into a state of alignment if need be. Once alignment is achieved, the system is zeroed to essentially nullify the accumulation of geometric errors present in any given collection of parts. Additional points of alignment between additional jig positions and the matching deflected nail state can be measured using the probe, and the relationship between this deflection and the zeroed state can be used to calibrate the rate of deflection with the position of the jig.

Persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

1. A probe for measuring deformation of an orthopedic implant implanted in a patient, comprising:
a body portion configured to be implanted into the orthopedic implant, the body portion being arranged to conform with deformations in the orthopedic implant; and
a deformation measuring element associated with the body portion in manner to measure deformation of the probe when inserted into an orthopedic implant.

2. The probe of claim 1, wherein deformation of the probe mimics deformation of the orthopedic implant.

3. The probe of claim 1, wherein the deformation measuring element comprises one or more strain gages disposed in one or more locations on the probe.

4. The probe of claim 1, wherein the probe is divided into a plurality of segments and the deformation measuring element is disposed to measure deformation of one of the plurality of segments.

5. The probe of claim 4, wherein the deformation measurement element comprises one or more deformation measurement elements associated with each of the plurality of segments, and wherein the measured deformation is integrateable to determine a composite deformation of the orthopedic implant.

6. The probe of claim 4, wherein each of the plurality of segments are demarcated with the demarcation elements configured to guide a portion of the probe so that the probe follows the same trajectory as the implant.

7. The probe of claim 1, wherein the probe is sized and shaped to displace relative to the orthopedic implant such that a single deformation measuring element yields multiple data points.

8. The probe of claim 7, wherein the probe is configured to measure deformation enabling various planes of deformation to be determined.

9. The probe of claim 7, wherein the probe is configured to measure deformation enabling radial deflection to be determined in order that curvature and/or trajectories can be calculated.

10. The probe of claim 1, wherein the body portion comprises a square cross-section.

11. The probe of claim 10, wherein the deformation measuring element comprises strain gauges disposed on opposing sides of the square body portion.

12. A method of measuring deformation to align an instrument with an orthopedic implant implanted in a patient, comprising:
providing a body portion of a probe in an orthopedic implant, the body portion being arranged to conform with deformations in the orthopedic implant; and
measuring deformation of the probe with a deformation measuring element.

13. The method of claim 12, comprising making adjustments to a targeting jig such that the orthopedic implant remains in alignment with the targeting jig when deflection is present in the orthopedic implant, the targeting jig, or both.

14. The method of claim 12, comprising dividing the body portion of the probe into segments; and wherein measuring deformation of the probe comprises measuring deformation of the segments in a piecewise manner.

15. The method of claim 14, comprising integrating the measured segments together to determine a composite deformation of the body portion of the probe.

16. The method of claim 12, comprising changing the position of the body portion of the probe relative to the orthopedic implant while measuring to yield multiple data points from a given deformation measuring element.

17. The method of claim 16, wherein changing the position of the body portion comprise rotating the body portion about an axis of the probe to obtain deformation information.

18. The method of claim 17, comprising using the obtain deformation information to determine various planes of deformation of the orthopedic implant.

19. The method of claim 16, wherein changing the position of the body portion comprises axially translating the body portion along an axis of the probe to obtain a collection of points representing radial deflection.

20. The method of claim 19, comprising using the collection of points to determine curvature or trajectory of the orthopedic implant.

21. The method of claim 20, comprising compensating for measured deflection of the orthopedic implant by adjusting a targeting jig to accurately target a certain feature of the orthopedic implant.

22. The method of claim 16, comprising compensating for measured deflection of the orthopedic implant by adjusting a targeting jig to accurately target a certain feature of the orthopedic implant.

23. The method of claim 22, wherein adjusting a targeting jig to accurately target a certain feature comprises adjusting the jig in more than one plane that intersects the orthopedic implant.

24. A method of aligning a jig with a feature of an implant implanted in a patient, comprising:
detecting deformation with a strain gage on an orthopedic implant; and
based on the detected deformation, calculating the actual deformation to accurately predict a location of a feature on the orthopedic implant while the implant is in a deformed state.

25. The method of claim 24, comprising aligning a jig with the feature on the orthopedic implant while the implant is in the deformed state taking into account the detected deformation.

26. The method of claim 24, wherein calculating the actual deformation comprises comparing detected deformation from a first probe associated with or incorporated into the implant and detected deformation from a second probe associated with or incorporated into the jig.

27. The method of claim 24, wherein the strain gage is disposed on a probe inserted into the orthopedic implant.

28. A system for determining the location of a feature of an implant and aligning a surgical instrument with the feature, the system comprising:
a measuring element configured to measure deformation of an orthopedic implant in a patient to identify a feature of the orthopedic implant in the patient; and
a jig dimensionally adjustable to match the measured deformation of the orthopedic implant to align with the feature of the orthopedic implant.

29. The system of claim 28, wherein the jig has one or more degrees of freedom such that the jig can be manipulated into a desired position and, wherein the jig is configured to be locked into the desired position.

30. The system of claim 29, wherein the one or more degrees of freedom are achieved using a series of sliding elements.

31. The system of claim 30, wherein the series of sliding elements comprise rectangular blocks in rectangular recesses.

32. The system of claim 31, wherein the rectangular blocks and rectangular recesses comprise a first rectangular block and rectangular recess and a second rectangular block and rectangular recess, with the first rectangular block and rectangular recess arranged orthogonal to the second rectangular block and rectangular recess.

33. The system of claim 30, wherein the sliding elements comprise a rectangular tongue in a first rectangular slot.

34. The system of claim 33, wherein the sliding elements comprise the rectangular tongue in a second rectangular slot.

35. The system of claim 28, wherein the jig comprises three adjustable struts operable to align a surgical instrument in three degrees of freedom.

36. The system of claim 35, comprising a processing system configured to output adjustment settings for the three adjustable struts to align a portion of the jig with the feature of the orthopedic implant.
